(19) United States
(12) Patent Application Publication
(10) Pub. No.: US 2023/0134456 A1
(43) Pub. Date: May 4, 2023

(54) ADAPTOR FOR ROBOTICALLY- GUIDED HIP CUP IMPACTION

(71) Applicants: Ajith Airody, L’Île-Bizard (CA); Jean-Francois Girouard, Terrasse-Vaudreuil (CA); Jeremie Menard, Montreal (CA); Cyril Tran, Brossard (CA)

(72) Inventors: Ajith Airody, L’Île-Bizard (CA); Jean-Francois Girouard, Terrasse-Vaudreuil (CA); Jeremie Menard, Montreal (CA); Cyril Tran, Brossard (CA)

(21) Appl. No.: 17/977,776

(22) Filed: Oct. 31, 2022

Related U.S. Application Data
(60) Provisional application No. 63/274,372, filed on Nov. 1, 2021.

(51) Int. Cl. A61F 2/46

(52) U.S. Cl. CPC A61F 2/4609; A61F 2002/4681

(57) ABSTRACT

An impaction adaptor connectable to a surgical drill and a surgical impactor can include a body comprising a proximal portion defining a body bore and including a first plurality of projections; and a distal portion connected to the proximal portion and insertable into the surgical impactor; a shaft located at least partially within the body bore and engageable with the surgical drill to be driven to rotate within the body bore; and a driving body located at least partially within the body bore and secured to the shaft, the driving body including a plurality of second projections rotatably engageable with the first projections to cause translation of the driving body relative to the body to deliver an impaction force to the surgical impactor in response to rotation of the shaft.

### CLAIM OF PRIORITY

This application claims the benefit of U.S. Provisional Patent Application Ser. No. 63/274,372, filed on Nov. 1, 2021, the benefit of priority of which is claimed hereby, and which is incorporated by reference herein in its entirety.

### BACKGROUND

During a hip arthroplasty procedure, an impactor can be used by a surgeon to help prepare the acetabular cup and the femur to receive an implant. For example, an impactor can be used to drive an acetabular implant into the acetabular cup or broach the femur to prepare an osseus envelope for receiving a femoral implant. An incision can be first made in the hip region of the patient, into which the impactor can be inserted to access a bone surface of the acetabulum or the femur. A surgeon can manually position the impactor proximal to such bone surface(s) by hand; or the impactor can be connected to a robotic arm to help the surgeon position and maintain the impactor proximal to the bone surface(s) during the hip arthroplasty procedure.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a perspective view of an adaptor operatively coupling a drill and an impactor.

FIG. 2A illustrates an isometric view of an adaptor.

FIG. 2B illustrates a side cross-sectional view of a proximal portion of an adaptor.

FIG. 3A illustrates an isometric view of a plurality of first projections of an adaptor.

FIG. 3B illustrates a top view of an adaptor.

FIG. 4A illustrates an isometric view of a shaft of an adaptor.

FIG. 4B illustrates an isometric view of driving body of an adaptor.

FIG. 5 illustrates an exploded view of an adaptor.

FIG. 6 illustrates a cross-sectional side view of an adaptor.

FIG. 7 illustrates a method of imparting an axial impaction force to a surgical impactor.

FIG. 8 illustrates a perspective view of a robotic surgical system.

FIG. 9 illustrates a schematic view of a robotic surgical system for robotically assisted impacting.

FIG. 10 illustrates a block diagram of an example machine upon which any one or more of the techniques discussed herein can be performed.

### DETAILED DESCRIPTION

A total hip replacement procedure, or total hip arthroplasty, can involve making an access incision in a hip region of a patient. Various surgical devices configured for intra-procedurally reaming, cutting, broaching, impacting, or otherwise preparing bone surfaces of a patient during total hip arthroplasty can be inserted through the incision, such as to access the proximal femur or the acetabular cup. Preparation of the proximal femur, such as the femoral head, often includes broaching the femur with an impactor, such as to create an osseous envelope for implant insertion by repeatedly striking the impactor with a mallet. Preparation of the acetabular cup often involves impacting the acetabular cup with the impactor, such as to insert or otherwise install an implant by repeatedly striking the impactor with a mallet.

However, manual impaction can be a time-consuming, challenging, and potentially hazardous operation for the surgeon. First, precisely positioning and maintaining the impactor in a location with respect to a bone of a patient, such as in accordance with a surgical plan, can be time-consuming. Second, carefully maintaining the impactor in a position aligned with a single axis while repeatedly striking the impactor with consistent force can be challenging and fatiguing. Third, manually striking the impactor by hand can result in repetitive stress injuries for the surgeon over time. Further, the several aspects of manual impaction discussed above can be difficult for a surgeon to learn, and various patient outcomes can be significantly diminished if any aspect of implantation is imprecisely or otherwise inadequately performed.

The present disclosure can help to address the above issues, among others, such as by providing an adaptor capable of operatively coupling an impactor to an existing motive source, such as a surgical drill configured to rotate various attachments, to thereby provide a repeating axial impaction force to the impactor. For example, a surgical drill, or other electrically or pneumatically powered surgical devices, are often used in addition to an impactor to cut, mill, or otherwise shape various bone surfaces during an arthroplasty procedure, such as by powering a rotatable cutting head of a reamer. The adaptor can include a portion receivable within the impactor, and a shaft engageable with the surgical drill to receive a rotational force therefrom. In response to rotation of the shaft via the surgical drill, the adaptor can transform the torque into a repeating axial impaction force deliverable to the impactor to help reduce the need for a surgeon to manually strike to the impactor to therefore reducing surgeon fatigue and repetitive stress injuries. The adaptor can thereby help to increase the consistency and predictability of the impaction force applied to a bone surface by an impactor, such as by reducing the variability inherent in manual mallet strikes delivered to the impactor by hand during a total hip arthroplasty procedure and concurrently helping to reduce patient movement, such as caused by an inconsistent impaction force.

Additionally, the impactor can be coupled to a robotic arm, such as to help reduce the length, and improve the precision, of a total hip arthroplasty procedure. For example, the robotic arm can help a surgeon improve the speed and accuracy at which the impactor can be positioned with respect to a bone in accordance with a preoperative surgical plan and concurrently reduce the amount of training necessary for a surgeon to adequately perform a total hip arthroplasty. The robotic arm can also help to improve the axial stability of the impactor, such as relative to a human hand, during implant impaction or insertion. The adaptor can also reduce the number of instrument components necessary to perform an arthroplasty procedure, such as by allowing for an increased commonality of parts between reaming and impaction steps of the procedure. For example, reaming heads, femoral broaches, and acetabular cups can attach to a common surgical device or shaft.

While the above and following examples are discussed in view of a hip arthroplasty procedure, the described adaptor, drill, impactor, and robotic arm can be utilized in other similar arthroplasty procedures, such as in knee or shoulder arthroplasty procedures.

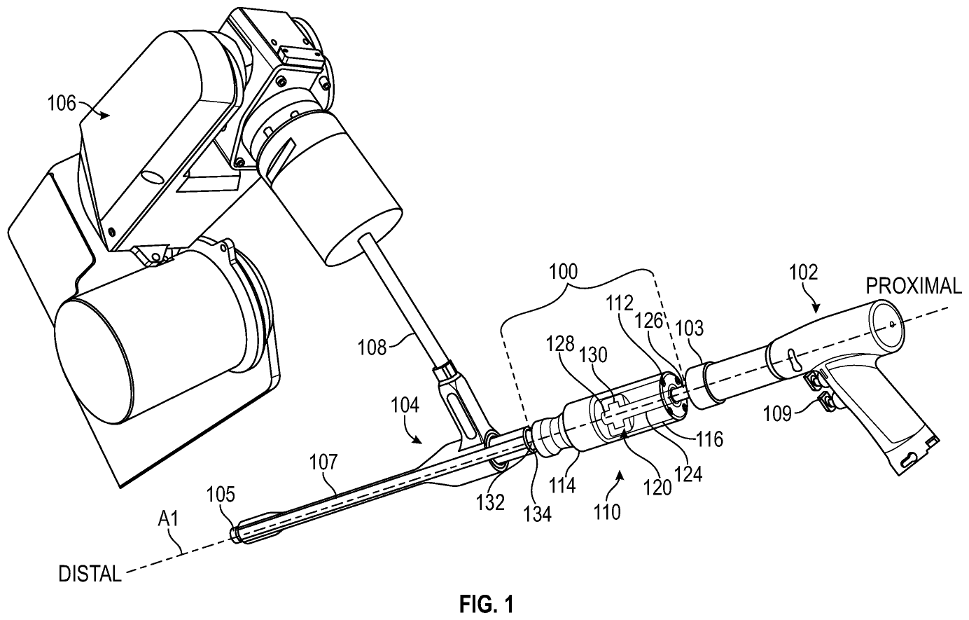

FIG. 1 illustrates a perspective view of an adaptor 100 operably coupling a drill 102 and an impactor 104, in accordance with at least one example of the present application. Also shown in FIG. 1 is a longitudinal axis A1, and orientation indicators Proximal and Distal relating to relative positions along the adaptor 100. The drill 102 can be a surgical drill, driver, reamer, or other powered surgical device operable to generate and output a rotational force. For example, the drill 102 can include a chuck 103, such as configured to engage with and rotate a rod or shaft. In one example, the drill 102 can be the Universal Power System from Zimmer Biomet Holdings, Inc.

The impactor 104 can be a manual surgical impactor, or other surgical devices configured to receive an axial impaction force, such via a mallet strike, to shape a surface of a bone. For example, the impactor 104 can include a head 105 configured to translate distally along the longitudinal axis A1 to impact or cut bone in response to receiving the axial impaction force, such from a rod 107 extending at least partially through the impactor 104 along the longitudinal axis A1 between the adaptor 100 and the head 105. The rod 107 can be translatable and rotatable within the impactor 104; and can be in contact with, or otherwise connected to, the head 105 to operatively couple the adaptor 100 or the drill 102 to the head 105. In such examples, the head 105 can be, for example, but not limited to, a femoral broach or a portion thereof, or a replacement implantable acetabular cup configured to be implanted into bone.

In some examples, the impactor 104 can be converted from a surgical device configured to receive an axial impaction force to a surgical device configured to receive a rotational force, such as to ream or otherwise shape a surface of a bone. In such examples, the head 105 configured to translate distally along the longitudinal axis A1 to impact or cut bone in response to receiving an axial impaction force from the rod 107 can be replaced with a head 105 configured to rotate around the longitudinal axis A1 to ream or cut bone in response to receiving a rotational force from the rod 107.

The impactor 104 can be coupled to a robotic arm 106. For example, the impactor 104 can be configured to engage with various types or styles of a pre-existing end effector coupler 108 connectable to the robotic arm 106. For example, the end effector coupler 108 can generally be a solid or a hollow shaft defining a square cross-sectional shape, but the end effector coupler 108 can also define circular, triangular, or rectangular cross-sectional shapes, or the like. In one example, the robotic arm 106 can be a 6 degree-of-freedom (DOF) robot arm, such as the ROSA® robot from Medtech, a Zimmer Biomet Holdings, Inc. company. The robotic arm 106 can adjust and maintain a position of the drill 102 and the impactor 104 before or during a surgical procedure. For example, the robotic arm 106 can be used to position the impactor 104 in a planned position, such as in accordance with a preoperative plan. The robotic arm 106 can help to control the position and movement of the impactor 104 relative to a patient more precisely and steadily than a human hand.

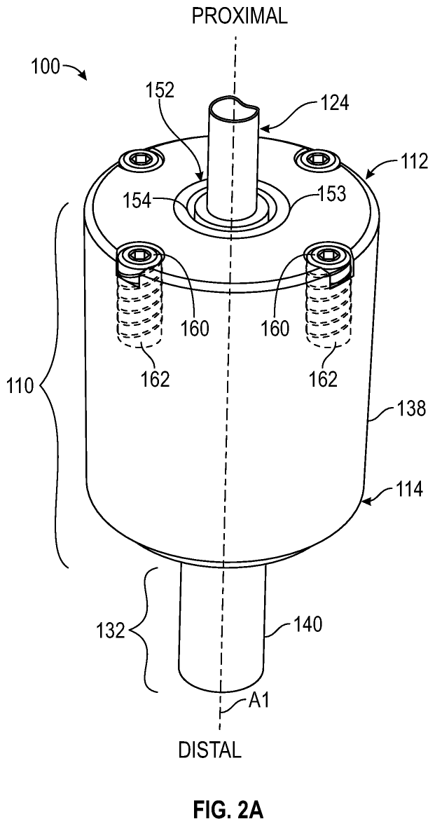

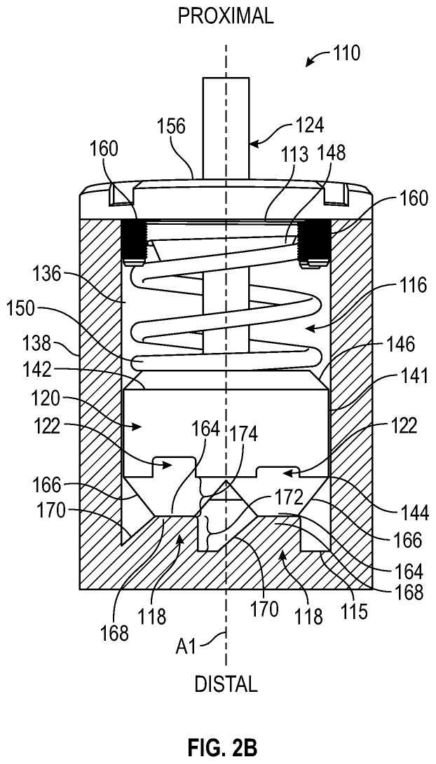

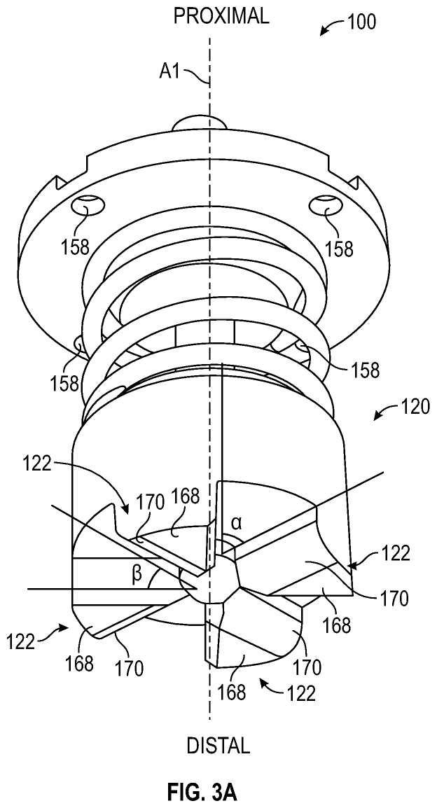

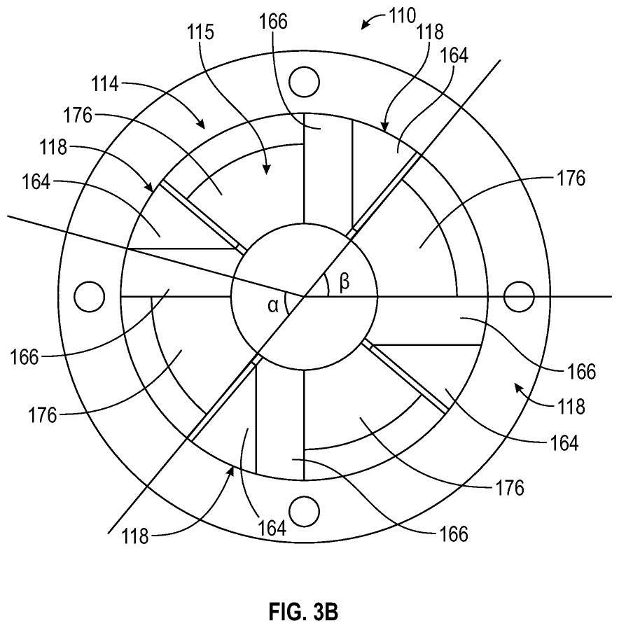

As shown in FIG. 1, the adaptor 100 can include a proximal portion 110 (shown in shadow in FIG. 1) defining a first end portion 112, a second end portion 114, a body bore 116 (shown in shadow in FIG. 1), a plurality of first projections 118 (FIGS. 2B & 3B), a driving body 120, a plurality of second projections 122 (FIGS. 2B, 3A, & 4B), a shaft 124 defining a first portion 126 and a second portion 128, a protrusion 130, and a distal portion 132. The proximal portion 110 can define the longitudinal axis A1 and the body bore 116. The body bore 116 can extend longitudinally through the proximal portion 110, such as between the first end portion 112 and the second end portion 114 along the longitudinal axis A1. The body bore 116 can generally be cylindrical in shape. The plurality of first projections 118 can extend proximally from the second end portion 114 into the body bore 116; and can form a radial arrangement around the longitudinal axis A1. The driving body 120 can be located within the body bore 116.

The shaft 124 can extend along the longitudinal axis A1. The first portion 126 and the second portion 128 can be opposite proximal and distal ends or segments, respectively, of the shaft 124. The first portion 126 of the shaft 124 can be located proximally to the first end portion 112 of the proximal portion 110. The first portion 126 of the shaft 124 can be configured to engage, such as by being at least partially receivable within, the drill 102. For example, the second portion 128 of the shaft 124 can be sized and shaped to be received within a pre-existing chuck 103 of the drill 102, to thereby receive a rotational force generated by the drill 102 upon activation of the drill 102.

The second portion 128 of the shaft 124 can be located within the body bore 116, such as in contact with or otherwise connected to, the second end portion 114 of the proximal portion 110. The shaft 124 can be configured to rotate the driving body 120, such as in response to activation of the drill 102. For example, the shaft 124 can include the protrusion 130. The protrusion 130 can generally be a body extending radially outward from the portion of the shaft 124 translatably received within the driving body 120. The protrusion 130 can engage the driving body 120 to rotate the driving body 120 in response to rotation of the shaft 124. The driving body 120 can include the second projections 122. The second projections 122 can extend distally from the driving body 120 toward the first projections 118; and can form a radial arrangement around the longitudinal axis A1.

The second projections 122 can be configured to translate the driving body 120 proximally and distally within the body bore 116 during rotation of the driving body 120. For example, the second projections 122 can be sized and shaped to rotatably engage the first projections 118, such that the driving body 120 repeatedly contacts the second end portion 114 of the proximal portion 110 to impart or transfer an axial impaction force to the impactor 104. The distal portion 132 can generally be a cylindrically shaped body connected to and extending distally from the proximal portion 110 along the longitudinal axis A1. In some examples, the distal portion 132 can be partially or completely recessed into the proximal portion 110. The distal portion 132 can be configured to engage, such as by being at least partially receivable within, the impactor 104.

For example, the distal portion 132 can be sized and shaped to extend into a channel 134 defined by the impactor 104. The channel 134 can extend partially or completely through the impactor 104 along the longitudinal axis A1. In some examples, the channel 134 can be configured to receive the rod 107. The channel 134 can be sized and shaped to enable the rod 107 to translate axially along the longitudinal axis A1, such in response to receiving an axial impaction force from the distal portion 132 of the adaptor 100, or rotate around the longitudinal axis A1, such as in response to receiving a rotational force from the chuck 103 of the drill 102. The adaptor 100 can thereby operably couple the drill 102 to the impactor 104 (e.g., convert a rotational force generated by the drill into an axial impaction force usable by the impactor).

During an arthroplasty procedure, various aspects of bone preparation or implant insertion, such as reaming, femoral broaching, or acetabular cup impaction can be performed using the adaptor 100, the drill 102, the impactor 104, or the robotic arm 106. In some examples, at the beginning of an arthroplasty procedure, the impactor 104 can be configured to support reaming operations by including a head 105 configured to receive a rotational force from the drill 102 to ream bone via rotation around the longitudinal axis A1. In such examples, a user can first actuate a trigger 109 of the drill 102, to cause the chuck 103 to rotate the rod 107 engaged thereby to rotate the head 105 connected thereto, to ream bone when the head 105 is positioned proximal to a bone surface of a patient.

Subsequently, or in other examples at the beginning of an arthroplasty procedure, a user can convert the impactor 104 from a surgical device configured to support reaming operations to a surgical device configured to support impaction operations by replacing the head 105 configured to receive a rotational force with a head 105 configured to receive an axial impaction force, decoupling or otherwise disengaging the chuck 103 of the drill 102 from the rod 107, inserting the distal portion 132 of the adaptor 100 into the channel 134 of the impactor 104 until the proximal portion 110 contacts the rod 107 received therein, and inserting the first portion 126 of the shaft 124 into the chuck 103 of the drill 102. In some procedures, a user can operate the robotic arm 106 to position the impactor 104 proximal to a bone of a patient, such as by placing the head 105 in contact with a surface of an implant to be impacted into the femur or the acetabular cup. In some procedures, the robotic arm 106 can further accurately retain the impactor 104 in such a position for an extended length of time.

The user can then activate the drill 102, such as by actuating the trigger 109 of the drill 102, to cause the chuck 103 of the drill to rotate the shaft 124. In turn, the shaft 124 can rotate the driving body 120 to cause the driving body 120 to repeatedly impact the second end portion 114 of the proximal portion 110 by virtue of the second projections 122 rotatably engaging the first projections 118. The proximal portion 110 and the distal portion 132 can collectively transfer the axial impaction force generated by the driving body 120 to the impactor 104, such as to cause the head 105 to translate distally to impact an implant. After the arthroplasty procedure, the user can remove the first portion 126 of the shaft 124 from the chuck 103 and the distal portion 132 from the channel 134 of the impactor 104. The adaptor 100, or various components thereof, can subsequently be cleaned and sterilized in an autoclave in preparation for a future arthroplasty procedure. The adaptor 100 can thereby help perform one or more operations of an arthroplasty procedure.

FIG. 2A illustrates an isometric view of an adaptor 100. FIG. 2B illustrates a side view of a proximal portion of an adaptor 100. Also shown in FIGS. 2A-2B is a longitudinal axis A1, and orientation indicators Proximal and Distal relating to relative positions along the adaptor 100. FIGS. 2A-2B are discussed below concurrently with reference to the adaptor 100 shown in and described with regard to FIG. 1 above. The adaptor 100 can include a first end surface 113, a second end surface 115, a proximal inner surface 136 (shown in shadow in FIG. 2B), a proximal outer surface 138 (shown in shadow in FIG. 2B), a distal outer surface 140, an outer surface 141, a proximal surface 142, a distal surface 144, a first taper 146, a second taper 148, a biasing element 150, an aperture 152, a proximal bearing 154, a cap 156, a plurality of apertures 158 (shown in FIG. 3A), a plurality of fasteners 160, a plurality of bores 162 (shown in shadow in FIG. 2A), first contacting surfaces 164, first angled surfaces 166, second contacting surfaces 168, second angled surfaces 170, a first radial extension 172, and a second radial extension 174.

The first end portion 112 (FIG. 2A) can define the first end surface 113 (FIG. 2B) and the second end portion 114 (FIG. 2A) can define the second end surface 115 (FIG. 2B). The first end surface 113 and the second end surface 115 can generally be opposite proximal and distal ends, respectively, of the body bore 116. For example, the first end surface 113 can extend transversely across the first end portion 112 orthogonally or the longitudinal axis A1 to partially enclose the body bore 116. The second end surface 115 can extend transversely across the second end portion 114 orthogonally or the longitudinal axis A1 to partially enclose the body bore 116.

The proximal portion 110 can include the proximal inner surface 136 and the proximal outer surface 138. The proximal inner surface 136 can be an inner surface of the proximal portion 110, such as a surface defined by the body bore 116. In some examples, the first projections 118 can extend radially from the proximal inner surface 136 into the body bore 116, such as toward the longitudinal axis A1. The proximal outer surface 138 can be an outer surface of the proximal portion 110. The distal portion 132 can include the distal outer surface 140. The distal outer surface 140 can be an outer surface of the distal portion 132. The proximal inner surface 136, the proximal outer surface 138, or the distal outer surface 140 can each generally form a cylindrical shape. In some examples, the proximal inner surface 136, the proximal outer surface 138, or the distal outer surface 140 can form various three-dimensional shapes, such as including, but not limited to, cuboids, triangular prisms, rectangular prisms, hexagonal prisms, octagonal prisms, or the like.

The proximal outer surface 138 can define a diameter greater than a diameter defined by the distal outer surface 140, such as to allow the proximal portion 110 to contact the impactor 104 to limit distal translation of the distal portion 132 within the channel 134 (FIG. 1) of the impactor 104 (FIG. 1). For example, the proximal outer surface 138 can define a diameter of about, but not limited to, 65-70 millimeters, 71-75 millimeters, 76-80 millimeters, or 81-85 millimeters, and the distal outer surface 140 can define a diameter of about, but not limited to, 10-12 millimeters, 13-15 millimeters, or 15-17 millimeters. The proximal inner surface 136 can guide proximal and distal translation of the driving body 120 within the proximal portion 110. For example, the driving body 120 can include the outer surface 141. The outer surface 141 can be an outer surface of the driving body 120. The outer surface 141 can be sized and shaped to contact the proximal inner surface 136 defined by the body bore 116, such as to guide the driving body 120 during proximal and distal translation of the driving body 120 within the body bore 116.

The driving body 120 can include the proximal surface 142 and the distal surface 144. The proximal surface 142 and the distal surface 144 can be opposite proximal and distal ends or segments of the driving body 120, such as relative to the longitudinal axis A1. The proximal surface 142 of the driving body 120 can define or otherwise include the first taper 146. The first end portion 112 of the proximal portion 110 can define or otherwise include the second taper 148. For example, the second taper 148 can extend distally into the body bore 116 from the first end surface 113. The first taper 146 and the second taper 148 can form, for example, but not limited to, a generally conical, trapezoidal, or triangular shape. The biasing element 150 can be, for example, but not limited to, a coil spring, a wave spring, or the like. The biasing element 150 can be configured, such as by being sized and shaped, to extend axially within the body bore 116.

The first taper 146 and the second taper 148 can be configured to support the biasing element 150 to axially align the biasing element 150 with the longitudinal axis A1 within the body bore 116. For example, the first taper 146 and the second taper 148 can concurrently contact and engage the biasing element 150, such as by extending longitudinally into at least a portion or length of the biasing element 150, relative to the longitudinal axis A1, to center the biasing element 150 within the body bore 116. The biasing element 150 can be configured to bias the driving body 120 distally within the body bore 116, such toward or against the second end surface 115 of the second end portion 114. For example, when the driving body 120 translates proximally within the body bore 116, the biasing element 150 can be compressed between the proximal surface 142 or the first taper 146 and the first end surface 113 of the first end portion 112 or the second taper 148. The spring tension of the biasing element 150 can then drive the driving body 120 distally within the body bore 116 to contact and deliver an axial impaction force to the second end surface 115 of the second end portion 114.

The first end portion 112 of the proximal portion 110 can define the aperture 152 and the inner surface 153. The aperture 152 can be a bore or opening extending transversely through the first end surface 113 of the first end portion 112 along the longitudinal axis A1. The inner surface 153 can be a surface defined by the aperture 152. The aperture 152 can be configured to receive at least a portion of the shaft 124. For example, the aperture 152 can be sized and shaped to allow the inner surface 153 to contact and maintain the shaft 124 in a position axially aligned with the longitudinal axis A1. In some examples, such as shown in FIG. 2A, the first end portion 112 can include the proximal bearing 154. The proximal bearing 154 can be a ball bearing, a needle bearing, a plain bearing, a bushing, or other friction reducing devices, such as surfaces configured to promote rotation. As such, the inner surface 153 can be configured to engage with various three-dimensional shapes defined by the shaft 124, such as a cylinder, or a cuboid, a triangular prism, rectangular prism, hexagonal prism, octagonal prism, or the like. The proximal bearing 154 can thereby reduce friction between the first end portion 112 of the proximal portion 110 and the shaft 124.

In some examples, the first end portion 112 of the proximal portion 110 can define or otherwise include the cap 156. The cap 156 can include the first end surface 113, the aperture 152, the inner surface 153, or the proximal bearing 154. The cap 156 can include a plurality of apertures 158 (FIG. 3A) extending transversely therethrough, such as parallel to and laterally offset from the longitudinal axis A1. Each of the plurality of apertures 158 can be configured to receive at least a portion of one of the plurality of fasteners 160. The proximal portion 110 can define a plurality of bores 162. The plurality of bores 162 can extend transversely and distally into the first end portion 112, such as parallel to and laterally offset from the longitudinal axis A1. Each of the plurality of bores 162 can be configured to receive at least a portion of one of the plurality of fasteners 160.

The apertures 158 and the bores 162 can be formed in complementary radial locations or orientations in the cap 156 and the proximal portion 110 respectively, such that the apertures 158 and the bores 162 are aligned when the cap 156 is positioned on first end portion 112 of the proximal portion 110. The fasteners 160 can thereby be inserted through the apertures 158 to engage the bores 162 to secure the cap 156 to the proximal portion 110. The adaptor 100 can be configured to define various numbers of the apertures 158 and the bores 162, such as based on the number of fasteners 160 the adaptor 100 includes. In one example, the adaptor 100 can include four of the apertures 158, four of the fasteners 160, and four of the bores 162. In other examples, the adaptor 100 can define or otherwise include, for example, but not limited to, two, three, five, or six of the apertures 158, the fasteners 160, and the bores 162.

The cap 156 can be configured to be removably secured to the first end portion 112 of the proximal portion 110. For example, each of the fasteners 160 and the bores 162 can define corresponding threads, such as to allow each of the fasteners 160 to threadably engage each of the bores 162 to removably couple the cap 156 to the proximal portion 110. In other examples, the cap 156 can be removably secured to the first end portion 112 with other types of fasteners 160. In some examples, the cap 156 can be fixedly secured to the proximal portion 110. For example, the fasteners 160 can be rivets, or the cap 156 can alternatively be secured to the proximal portion 110 by welding, adhesives, or the like. The first projections 118 can include the first contacting surfaces 164 and the first angled surfaces 166. Each of the first contacting surfaces 164 can be a proximal surface defined by each of the first projections 118. Each of the first angled surfaces 166 can be a surface extending between each of the first contacting surfaces 164 and the second end portion 114 of the proximal portion 110. The second projections 122 can include the second contacting surfaces 168 and the second angled surfaces 170. Each of the second contacting surfaces 168 can be a surface defined by each of the second projections 122. Each of the second angled surfaces 170 can be a surface extending between each of the second contacting surfaces 168 and the distal surface 144 of the driving body 120. The first angled surfaces 166 and the second angled surfaces 170 can be configured to correspond to one another to enable the driving body 120 to translate proximally and distally within the body bore 116 via rotational engagement between each projection of the first projections 118 and each projection of the second projections 122.

For example, during rotation of the driving body 120 in response to rotation of the shaft 124, the second angled surfaces 170 can contact and engage, such as by translating or sliding vertically and laterally along, the first angled surfaces 166 to cause the driving body 120 to translate proximally until the second contacting surfaces 168 engage, such as by translating or sliding laterally along, the first contacting surfaces 164. The second angled surfaces 170 can then contact and engage, such as by vertically and laterally along, the first contacting surfaces 164, to cause the driving body 120 to translate distally until the distal surface 144 of the driving body 120 contacts the second end surface 115 of the second end portion 114. In one example, such as shown in FIG. 2B, each the first projections 118 can define one of the first angled surfaces 166 and each of the second projections 122 can define two of the second angled surfaces 170. In other examples, each the first projections 118 can define two of the second angled surfaces 170 and each of the second projections 122 can define two of the second angled surfaces 170. The driving body 120 can thereby translate proximally and distally within the body bore 116 in response to rotation of the shaft 124, to impart or deliver an axial impaction force to the second end surface 115 of the second end portion 114 upon contact with the second end surface 115.

As shown in FIG. 2B, the first contacting surfaces 164 can define the first radial extension 172. The first radial extension 172 can be a linear distance, such as measured parallel to the longitudinal axis A1 between the second end surface 115 of the proximal portion 110 and each of the first contacting surfaces 164. For example, the first radial extension 172 can be the distance the first projections 118 extend proximally into the body bore 116 from the second end surface 115. The second contacting surfaces 168 can define a second radial extension 174. The second radial extension 174 can be a linear distance, such as measured parallel to the longitudinal axis A1 between the distal surface 144 of the driving body 120 and the second contacting surfaces 168. For example, the second radial extension 174 can be the distance the second projections 122 extend distally into the body bore 116 from the driving body 120 from the distal surface 144.

The first radial extension 172 and the second radial extension can be, for example, but not limited to, 6-7 millimeters or 8-9 millimeters. The first radial extension 172 can be sufficient to ensure that the second contacting surfaces 168 can impact or otherwise contact the second end surface 115 of the second end portion 114 during rotation of the driving body 120. The first radial extension 172 can be configured to be similar or different relative to the second radial extension 174. In some examples, the first radial extension 172 can be less than the second radial extension 174, such as to help ensure the second contacting surfaces 168 impact the second end surface 115 of the second end portion 114 before the first contacting surfaces 164 limit further distal translation of the driving body 120 within the body bore 116.

FIG. 3A illustrates a side view of a plurality of first projections 118 of an adaptor 100, in accordance with at least one example of the present application. FIG. 3B illustrates a top view of a plurality of second projections 122 of an adaptor 100, in accordance with at least one example of the present application. Also shown in FIG. 3A is a longitudinal axis A1, and orientation indicators Proximal and Distal relating to relative positions along the adaptor 100. FIGS. 3A-3B are discussed below concurrently with reference to the adaptor 100 shown in and described with regard to FIGS. 1-2B above.

The first contacting surfaces 164 of the first projections 118 can extend parallel to the second end portion 114 of the proximal portion 110 and orthogonally to the longitudinal axis A1. The second contacting surfaces 168 of the second projections 122 can extend parallel to the distal surface 144 of the driving body 120 and orthogonally to the longitudinal axis A1. In some examples, the first contacting surfaces 164 and the second contacting surfaces 168 can extend at various other angles relative to the longitudinal axis A1, such as about, but not limited, to 10-30 degrees, 31-50 degrees, or 51-70 degrees relative to the longitudinal axis A1. The first contacting surfaces 164 and the second contacting surfaces 168 can extend at complementary substantially identical or angles relative to one another or to the longitudinal axis A1, such as to help facilitate rotational engagement (e.g., vertical or lateral translation along) therebetween.

The first projections 118 and the second projections 122 can each include various numbers of individual projections. In one example, such as shown in FIGS. 3A-3B, the first projections 118 and the second projections 122 can each include four projections. In other examples, the first projections 118 and the second projections 122 can also include three, five, or six projections. Each of the first contacting surfaces 164 of the first projections 118 and the second contacting surfaces 168 of the second projections 122 can be radially spaced depending on the specific number of individual projections each of the first projections 118 and the second projections 122 include. The angle α can represent the radial spacing of the first projections 118 and the second projections 122.

For example, the first projections 118 and the second projections 122 each include three projections, the angle α between each of the first contacting surfaces 164 or the second contacting surfaces 168 can be about 97.38 degrees. If the first projections 118 and the second projections 122 include four projections, the angle α between each of the first contacting surfaces 164 or the second contacting surfaces 168 can about 67.38 degrees. If the first projections 118 and the second projections 122 each include five projections, the angle α between each of the first contacting surfaces 164 or the second contacting surfaces 168 can about 49.37 degrees. If the first projections 118 and the second projections 122 each include six projections, the angle α between each of the first contacting surfaces 164 or the second contacting surfaces 168 can about 37.3 degrees.

Each of the first angled surfaces 166 can form an angled, beveled, chamfered, concave, convex, or the like, shape between the first contacting surfaces 164 and the second end portion 114 of the proximal portion 110. Each of the second angled surfaces 170 can form angled, beveled, chamfered, concave, convex, or the like, shapes between the second contacting surfaces 168 and the distal surface 144 of the driving body 120. In one example, each of the first angled surfaces 166 can form a concave shape and each the second angled surfaces 170 can form a chamfered shape. In another example, each of the first angled surfaces 166 and each of the second angled surfaces 170 can form a beveled or chamfered shape.

The adaptor 100 can include the gaps 176. The gaps 176 can radially or laterally space the first projections 118 and the second projections 122. For example, the gaps 176 can be defined as the circumferential or angular space between each of the first angled surfaces 166 and an adjacent one of the first contacting surfaces 164 or an adjacent one of the second contacting surfaces 168. Angle β can represent the radial spacing of the gaps 176. The gaps 176 can form a variety of different spacings depending on the dimensions of the first angled surfaces 166 or the second angled surfaces 170. As such, the angle β can generally be less than the angle α.

The gaps 176 can also form a variety of different spacings depending on the number of projections the first projections 118 and the second projections 122 include. For example, if the first projections 118 and the second projections 122 of projections each include three projections, the angle β can be about 80 degrees. If the first projections 118 and the second projections 122 each include four projections, the angle β can be about 50 degrees. If the first projections 118 and the second projections 122 each include five projections, the angle β can be about 32 degrees. If the first projections 118 and the second projections 122 each include six projections, the angle β can be about 20 degrees.

In some examples, the angle β formed by the first angled surfaces 166 can be less than the angle β formed by the second angled surfaces 170, such as to help improve the rotational force required to cause proximal and distal translation of the driving body 120. For example, if the angle β is decreased, the driving body 120 can travel the linear distance defined by the first radial extension 172 over a longer period of time or a greater circumferential rotation, such as to thereby reduce the rotation force required to cause the driving body 120 to translate proximally or distally between the second end surface 115 of the second end portion 114 of the proximal portion 110 and the first contacting surfaces 164 of the first projections 118.

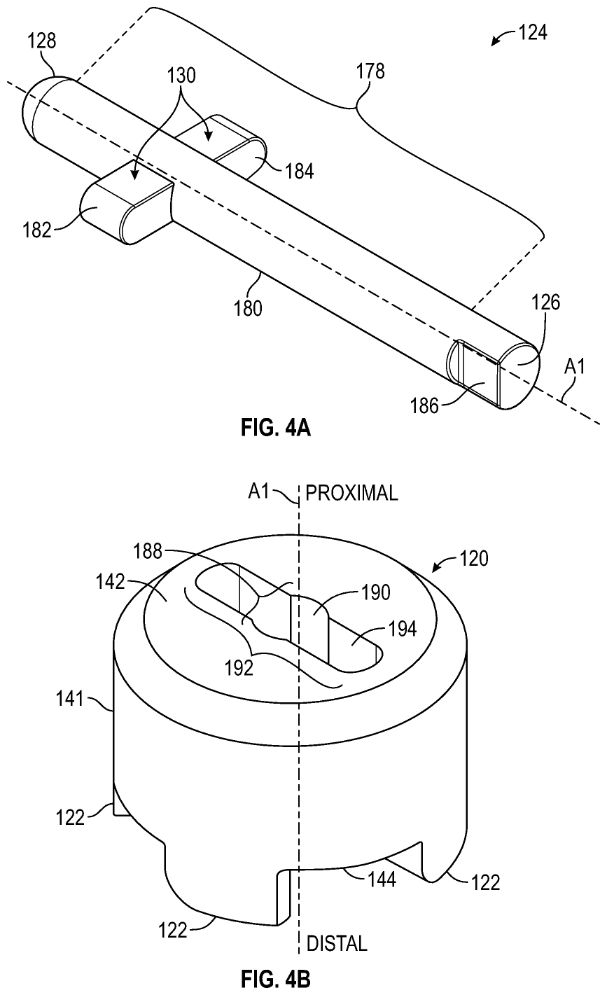

FIG. 4A illustrates an isometric view of a shaft 124 of an adaptor 100, in accordance with at least one example of the present application. Also shown in FIG. 4A is a longitudinal axis A1, and orientation indications Proximal and Distal relating to relative positions along the shaft 124. The shaft 124 can include a body portion 178, a body surface 180, a first protrusion 182, a second protrusion 184, and a facet 186 (and the first portion 126, the second portion 128, and the protrusion 130). The body portion 178 can be a length or segment of the shaft 124 extending between the first portion 126 and the second portion 128. The body surface 180 can be an outer surface of the shaft 124. The body surface 180 of the shaft 124 can form a generally cylindrical shape. In some examples, the body surface 180 can form other three-dimensional shapes, such as, but not limited to, a triangular prism, a rectangular prism, a hexagonal prism, an octagonal prism, or the like.

The protrusion 130 can extend radially outwardly from the body surface 180 of the shaft 124. The protrusion 130 can form a generally ellipsoidal shape. In some examples, the protrusion 130 can form other three-dimensional shapes such as, but not limited to, a triangular prism, a rectangular prism, a hexagonal prism, octagonal prism, or the like. The protrusion 130 can include various numbers of individual protrusions, such as, but not limited to, one, two three, four, five, or six protrusions extending outwardly from the shaft 124. In one example, such as shown in FIG. 4A, the protrusion 130 can include a first protrusion 182 and a second protrusion 184. The first protrusion 182 and the second protrusion 184 can be extend outwardly from the body surface 180 in various circumferentially offset positions relative to each other, such at 90 degrees, 180 degrees, or 270 degrees offset relative to each other. The first protrusion 182 and the second protrusion 184 can alternatively extend outwardly from the body surface 180 at other circumferentially offset positions relative to each other, such as at about, but not limited to, 20-60 degrees, 61-100 degrees, 101-140 degrees, or 141-180 degrees.

The first portion 126 of the shaft 124 can define or otherwise include the facet 186. The facet 186 can be a flattened or planer surface of the first portion 126. The facet 186 can be configured to help prevent relative rotation between the shaft 124 and the drill 102 (FIG. 1). For example, the facet 186 can be configured to engage a portion of the chuck 103 (FIG. 1) of the drill 102 to prevent relative rotation therebetween. The second portion 128 of the shaft 124 can define or otherwise include various three-dimensional shapes. In one example, such as shown in FIG. 4A, the second portion 128 can form a hemispherical or semi-hemispherical shape. In other examples, the second portion 128 can form a flattened or planer two-dimensional shape, or other three-dimensional shapes such as, but not limited to, a triangular prism, a cuboid, a rectangular prism, a hexagonal prism, an octagonal prism, or the like.

FIG. 4B illustrates an isometric view of driving body 120 of an adaptor 100, in accordance with at least one example of the present application. Also shown in FIG. 4B is a longitudinal axis A1, and orientation indicators Proximal and Distal relating to relative positions along the driving body 120. As shown in FIG. 4B, the driving body 120 can define a shaft bore 188, a bore surface 190, a slot 192, and a slot surface 194. The shaft bore 188 can extend through the driving body 120 between the proximal surface 142 and the distal surface 144 along the longitudinal axis A1. The shaft bore 188 can define the bore surface 190.

The bore surface 190 can be configured to contact and receive at least a portion or segment of the body surface 180 (FIG. 4A) of the shaft 124 (FIG. 4A). For example, the shaft bore 188 can be sized and shaped such that the bore surface 190 can translatably engage (e.g., can translate vertically and laterally along) the body surface 180 of the shaft 124 when the shaft 124 is positioned within the shaft bore 188. The slot 192 can extend through the proximal surface 142 of the driving body 120. The slot 192 can extend within the driving body 120 at least partially between the proximal surface 142 and the distal surface 144. The slot 192 can intersect the shaft bore 188. For example, the slot 192 can extend generally orthogonally to the longitudinal axis A1 and transversely through the shaft bore 188.

The slot 192 can define the slot surface 194. The slot surface 194 can be configured to contact and receive the protrusion 130 (FIG. 4A), such as including the first protrusion 182 (FIG. 4A) and the second protrusion 184 (FIG. 4A). For example, the slot 192 can be sized and shaped such that the slot surface 194 can translatably engage (e.g., can translate vertically and laterally along) the first protrusion 182 and the second protrusion 184 when protrusion 130 is positioned within the slot 192. When the shaft 124 is positioned within the shaft bore 188 of the driving body 120, the second portion 128 of the shaft 124 can extend distally beyond the distal surface 144 of the driving body 120, such as shown in FIGS. 2B and 3A. In some examples, the second portion 128 can contact the second end surface 115 of the second end portion 114 of the proximal portion 110 when the shaft 124 is positioned within the shaft bore 188.

In view of the above, when the shaft 124 receives a rotational force, the first protrusion 182 and the second protrusion 184 can engage the slot surface 194 to rotate the driving body 120. In turn, the second projections 122 of the driving body 120 can engage the first projections 118 to cause proximal and distal translation of the driving body 120. During proximal and distal translation of the driving body 120, the bore surface 190 can translate vertically and laterally along the body surface 180 of the shaft 124, and the slot surface 194 can concurrently translate vertically and laterally along first protrusion 182 and the second protrusion 184. The shaft bore 188 and the slot 192 can thereby enable the shaft 124 to rotate the driving body 120 while concurrently allowing the driving body 120 to translate proximally and distally relative to the shaft 124.

The adaptor 100, including any of various components thereof shown in and described above with regard to FIGS. 1-4B, such as the proximal portion 110, the distal portion 132, the driving body 120, the shaft 124, or the biasing element 150, can be made from, but not limited to, plastics, composites, rubber, or ceramics. For example, the components listed above can be molded, printed, or otherwise made from, ABS plastic. In other examples, the adaptor 100, including any of various components thereof shown in and described above with regard to FIGS. 1-4B, such as the proximal portion 110, the distal portion 132, the driving body 120, the shaft 124, or the biasing element 150, can be made from, but not limited to, can also each be made from stainless steel, aluminum, or other metals via machining or metallic molding.

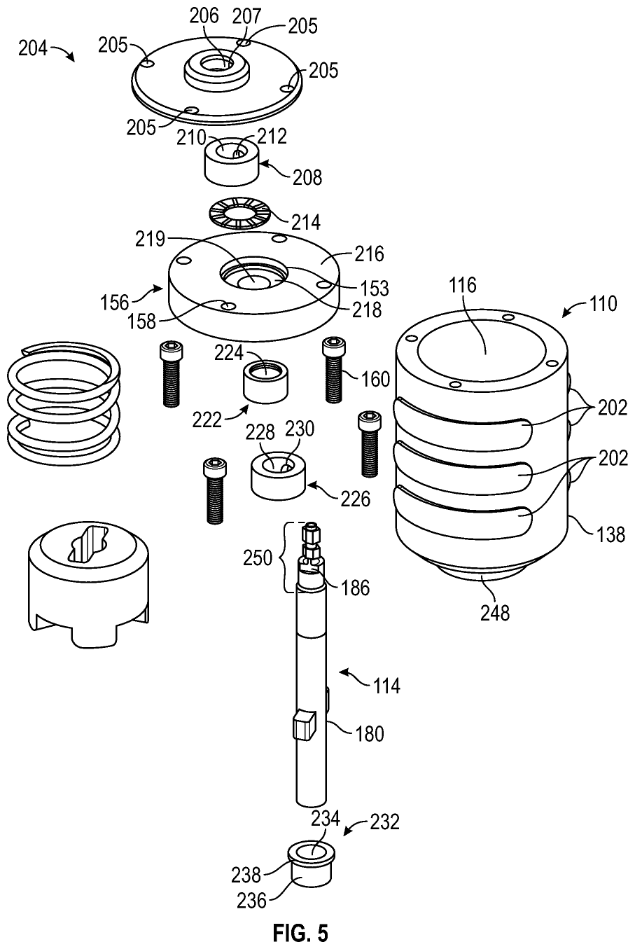

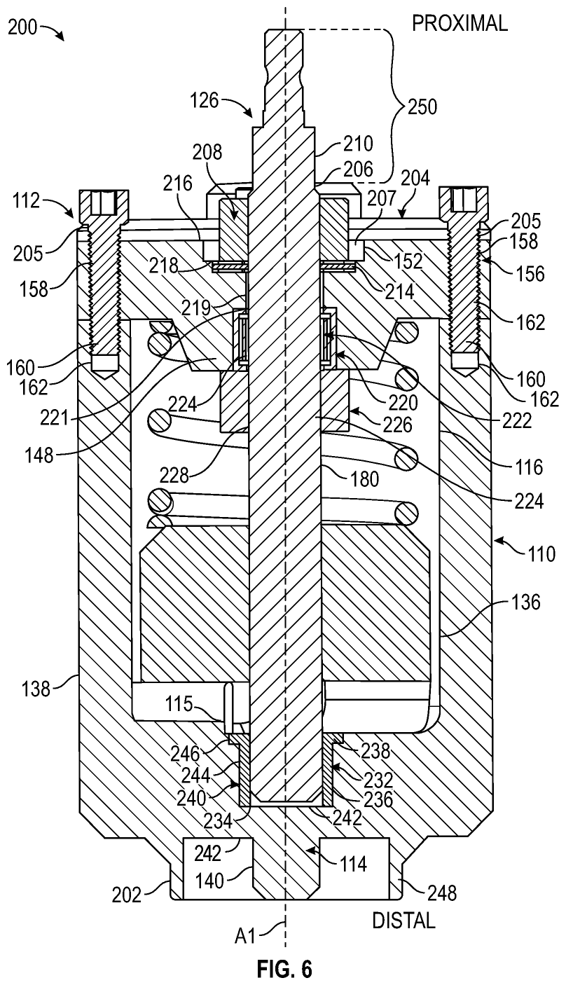

FIG. 5 illustrates an exploded view of an adaptor 200. FIG. 6 illustrates a cross-section of an adaptor 200. FIG. 6 illustrates a cross-sectional side view of an adaptor 200. Also shown in FIG. 6 is a longitudinal axis A1, and orientation indicators Proximal and Distal relating to relative positions along the adaptor 200. FIGS. 5-6 are discussed below concurrently with reference to the adaptor 100 shown in and described with regard to FIGS. 1-4B above. The adaptor 200 can be similar to the adaptor 100, at least in that the adaptor 200 can include any elements or components of the adaptor 100. As shown in FIGS. 5-6, the adaptor 200 can include grip features 202 (FIG. 5), a top plate 204, a plurality of second apertures 205, a first shaft bore 206, a collar recess 207 (FIG. 6), a first collar 208, a first collar surface 210, a first retainer 212 (FIG. 5), a bearing 214, a top surface 216, a first surface 218, a second shaft bore 219, a second aperture 220, a second surface 221 (FIG. 6), a first bushing 222, a first bushing surface 224, a second collar 226, a second collar surface 228, a second retainer 230, a second bushing 232, a second bushing surface 234, an outer surface 236, a flange 238, a shaft recess 240 (FIG. 6), a distal surface 242 (FIG. 6), a first portion 244 (FIG. 6), a second portion 246 (FIG. 6), an extension 248, and a bit portion 250.

The grip features 202 can be protrusions or projections extending radially outwardly from the proximal outer surface 138 of the proximal portion 110. The grip features 202 can form various three-dimensional shapes such as, but not limited to, an ellipsoid, a triangular prism, a rectangular prism, a hexagonal prism, octagonal prism, or the like. In one example, such as shown in FIG. 5, the grip features 202 can collectively include six of the grip features 202. In other examples, the grip features 202 can collectively include other numbers of individual grip features, such as, but not limited to, one, two, three, four, five, seven, eight, nine, or ten of the grip features 202. Each of the grip features 202 can extend outwardly from the proximal outer surface 138 in various parallel, non-parallel, or circumferentially offset positions relative to one another, such at 90 degrees, 180 degrees, or 270 degrees offset relative to one another. The grip features 202 can help a user hold or otherwise engage the proximal outer surface 138 of the adaptor 200, such as to limit relative rotation of the proximal portion relative to the shaft 124 during rotation of the shaft 124.

The first end portion 112 (FIG. 6) of the proximal portion 110 can include the cap 156 and the top plate 204. The top plate 204 can define the second apertures 205. The second apertures 205 can extend transversely through the top plate 204, such as parallel to and laterally offset from the longitudinal axis A1. Each of the second apertures 205 can be configured to receive at least a portion of one of the fasteners 160. The second apertures 205, the apertures 158, and the bores 162 can be formed in complementary radial locations or orientations in the top plate 204, the cap 156, and the proximal portion 110 respectively, such that the second apertures 205, the apertures 158, and the bores 162 can be aligned when the top plate 204 and the cap 156 are positioned on the proximal portion 110. The fasteners 160 can thereby be inserted through the second apertures 205 and the apertures 158 to engage the bores 162 to secure the top plate 204 and the cap 156 to the proximal portion 110.

The top plate 204 can define the first shaft bore 206. The first shaft bore 206 can be a bore or opening extending transversely through the top plate 204, such as along the longitudinal axis A1 (FIG. 6). The first shaft bore 206 can be configured to receive a portion of the shaft 124. For example, the first shaft bore 206 can be sized and shaped such that the body surface 180 of the shaft 124 can engage the first shaft bore 206 when a portion of the shaft 124 is positioned within the first shaft bore 206. The collar recess 207 can be a bore or opening extending transversely into and partially through the top plate 204. The collar recess 207 can be sized and shaped to receive at least a portion of the first collar 208. The first collar 208 can include the first collar surface 210 and the first retainer 212. The first collar surface 210 can be configured to contact and receive the shaft 124. For example, the first collar surface 210 can be sized and shaped to engage the body surface 180 of the shaft 124 when a portion of the shaft 124 is positioned within the first collar 208. The first retainer 212 can be a screw, a pin, a detent, a lock, or other devices or fixation methods.

The first retainer 212 can be configured to engage the body surface 180 of the shaft 124 when the shaft 124 is positioned at least partially within the first collar 208. For example, the first retainer 212 can extend transversely through the first collar 208 and inwardly beyond the first collar surface 210, such as by threadably engaging a portion of the first collar 208, to contact the body surface 180 of the shaft 124. The first retainer 212 can thereby prevent relative rotation between the first collar 208 and the shaft 124 and help to locate the shaft 124 within the body bore 116, such as by limiting vertical translation of the shaft 124 relative to the top plate 204. The bearing 214 can be a ball bearing, a needle bearing, a plain bearing, a bushing, or other friction reducing devices, such as surfaces configured to promote rotation.

The cap 156 can define the aperture 152, the inner surface 153, the top surface 216, the first surface 218, the second shaft bore 219, the second aperture 220, and the second surface 221. The top surface 216 can be configured to contact or otherwise interface with the top plate 204 when the top plate 204 is secured to the cap 156 and the proximal portion 110. The first surface 218 can be a distal end surface of the aperture 152, such as distally offset from the top surface 216 of the cap 156. The aperture 152 can be configured to at least partially receive the first collar 208 and the bearing 214. The first surface 218 can be configured to contact and support the bearing 214. As such, the bearing 214 can be positioned within the aperture 152 between the first collar 208 and the first surface 218. The bearing 214 can thereby promote rotation and reduce friction between the first collar 208 and the cap 156.

The second shaft bore 219 can be a bore or opening extending transversely through the cap 156, such as along the longitudinal axis A1 and concentrically with the aperture 152. The second shaft bore 219 can define a smaller or reduced diameter relative to the aperture 152. The second shaft bore 219 can be configured to receive a portion of the shaft 124. For example, the second shaft bore 219 can be sized and shaped such that the body surface 180 of the shaft 124 can engage the second shaft bore 219 when a portion of the shaft 124 is positioned within the second shaft bore 219, such as to promote rotation therebetween and reduce friction between the body surface 180 and second shaft bore 219. The second aperture 220 can be a bore or opening extending transversely into and partially through the cap 156, such as proximally into the second taper 148 (FIG. 6) along the longitudinal axis A1. The second surface 221 can be a proximal end surface of the second aperture 220, such as proximally offset from the second taper 148 of the cap 156.

The second aperture 220 can be configured to at least partially receive the first bushing 222. The second surface 221 can be configured to contact the first bushing 222 to help position the first bushing 222 along the shaft 124, such as by limiting vertical translation of the first bushing 222 relative to the cap 156. The first bushing 222 can define the first bushing surface 224. The first bushing surface 224 can be configured to contact and receive the shaft 124. For example, the first bushing surface 224 can be sized and shaped to engage the body surface 180 of the shaft 124 when a portion of the shaft 124 is positioned within the first bushing 222. The second collar 226 can include the second collar surface 228 and the second retainer 230. The second collar surface 228 can be configured to contact and receive the shaft 124. For example, the second collar surface 228 can be sized and shaped to engage the body surface 180 of the shaft 124 when a portion of the shaft 124 is positioned within the second collar 226. The second retainer 230 can be a screw, a pin, a detent, a lock, or other devices or fixation methods. The second retainer 230 can be configured to engage the body surface 180 of the shaft 124 when the shaft 124 is positioned at least partially within the second collar 226. For example, the second retainer 230 can extend transversely through the second collar 226 and inwardly beyond the second collar surface 228, such as by threadably engaging a portion of the second collar 226, to contact the body surface 180 of the shaft 124. The second retainer 230 can thereby prevent relative rotation between the second collar 226 and the shaft 124 and help to locate the shaft 124 within the body bore 116, such as by limiting vertical translation of the shaft 124 relative to the second taper 148 of the cap 156.

The second bushing 232 can include the second bushing surface 234, the outer surface 236, and the flange 238. The second bushing surface 234 can be configured to contact and receive the shaft 124. For example, the second bushing surface 234 can be sized and shaped to engage the body surface 180 of the shaft 124 when a portion of the shaft 124 is positioned within the second bushing 232, such as to promote rotation therebetween. The flange 238 can be a protrusion or projection extending circumferentially outwardly beyond the outer surface 236, such as orthogonally to the longitudinal axis A1. The proximal portion 110 can define the shaft recess 240. The shaft recess 240 can extend transversely through the second end surface 115 along the longitudinal axis A1.

The shaft recess 240 can define the distal surface 242, the first portion 244, and the second portion 246. The distal surface 242 can be a distal end surface of the shaft recess 240, such as distally offset from the second end surface 115. The first portion 244 can define a smaller diameter relative to the second portion 246. The first portion 244 and the second portion 246 of the shaft recess 240 can be configured to contact and receive the second bushing 232. For example, the first portion 244 can be sized and shaped to engage the outer surface 236 of the second bushing 232 and, such as to prevent relative rotation therebetween. The second portion can be sized and shaped to engage the flange of the second bushing 232. As such, the second bushing 232 can be positioned within the shaft recess 240 between the second end surface 115 and the distal surface 242 of the proximal portion 110. The second bushing 232 and the shaft recess 240 can thereby help to position the shaft 124 during rotation of the shaft 124, such as by limiting lateral translation relative to the proximal portion 110.

The distal portion 132 can include the extension 248. The extension 248 can extend distally from the proximal portion 110, such as parallel to the longitudinal axis A1 and the distal portion 132 (FIG. 6). For example, the extension 248 can extend distally beyond the distal portion 132, or the extension 248 can end at a location proximal to a distal-most or end surface of the distal portion 132. The extension 248 can define various three-dimensional shapes, such as circumferentially or otherwise laterally encompassing the distal portion 132. In some examples, such as shown in FIGS. 5-6, the first portion 126 (FIG. 6) of the shaft 124 can define or otherwise include the bit portion 250. The bit portion 250 can include the facet 186 (FIG. 5). The bit portion 250 can be configured to help prevent relative rotation between the shaft 124 and the drill 102 (FIG. 1). For example, the bit portion 250 can be configured, such as by being sized and shaped, to engage a portion of the chuck 103 (FIG. 1) of the drill 102 to help prevent relative rotation therebetween.

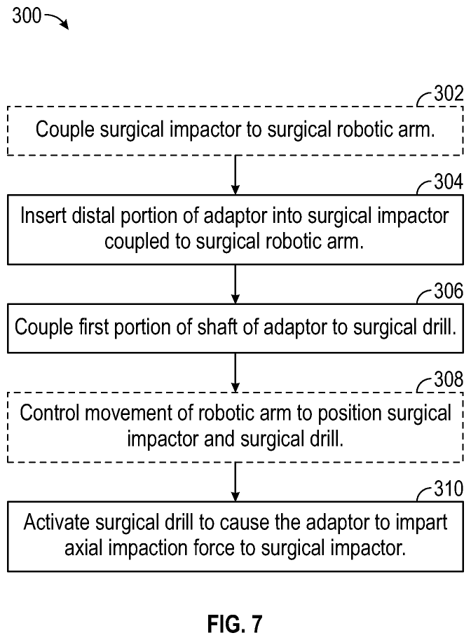

FIG. 7 illustrates a method 300 of imparting an axial impaction force to a surgical impactor, in accordance with one example of the present application. The steps or operations of the method 300 are illustrated in a particular order for convenience and clarity; many of the discussed operations can be performed by multiple different actors, devices, or systems. It is understood that subsets of the operations discussed in the method 300 can be attributable to a single actor, device, or system and can be considered a separate standalone process or method.

The method 300 can optionally begin with operation 302. The operation 302 can include coupling the surgical impactor to a surgical robotic arm. For example, a portion of the surgical impactor can be configured to be engageable with an end effector coupler extending from the surgical robotic arm. A user can thereby connect the end effector coupler of the surgical robotic arm to the portion of the surgical impactor configured to engage therewith, such as to allow a user to selectively or otherwise removably couple the surgical impactor to the robotic arm in preparation for, or after, an arthroplasty procedure.

The method 300 can include operation 304. The operation 304 can include inserting a distal portion of an adaptor into a surgical impactor coupled to a surgical robotic arm. For example, the surgical impactor can define a channel extending at least partially therethrough along a longitudinal axis. The distal portion of the adaptor can be configured to be insertable into the channel defined by the surgical impactor, such as to allow the adaptor to be at least partially received therein. A proximal portion of the adaptor can define a diameter greater than a diameter defined by the distal portion of the adaptor to limit distal translation of the adaptor within the channel of the surgical impactor, such as to locate the adaptor with respect to the surgical impactor.

The method 300 can include operation 306. The operation 306 can include coupling a first portion of a shaft of the adaptor to a surgical drill. For example, the adaptor can include a proximal portion including a shaft extending proximally therefrom along a longitudinal axis. A first portion of the shaft can be configured to be receivable within, or otherwise engage with, a portion of the surgical drill, such as via insertion thereinto by the user, to allow the shaft to receive a rotational force from the surgical drill. In some examples, the surgical drill can include a chuck configured engage the first portion of the shaft to prevent relative rotation between the chuck and the first portion of the shaft of the adaptor during rotation of the chuck.

The method 300 can optionally include operation 308. The operation 308 can include controlling movement of the robotic arm to position the surgical impactor proximal and the surgical drill. For example, the user can, such as via one or more user inputs to a user interface, cause the robotic arm to position at least a portion of the surgical impactor within an incision made in a hip region of a patient, such as to engage the surgical impactor with an acetabular implant during a hip arthroplasty procedure.

The method 300 can optionally include operation 310. The operation 310 can include activating the surgical drill to cause the adaptor to impart an axial impaction force to the surgical impactor. For example, the user can engage a trigger of the surgical drill, or can otherwise cause the surgical drill, to rotate the shaft of the adaptor engaged therewith around a longitudinal axis to cause the adaptor to impart a repetitive axial impaction force to the surgical impactor along the longitudinal axis. In turn, the axial impaction force imparted to the surgical impactor be transferred to an implant, such as to be inserted into the acetabular cup or the femur. In some examples, the trajectory can be based on a preoperative plan, such as based on a diagnostic image of the bone to determine the trajectory for implant insertion.

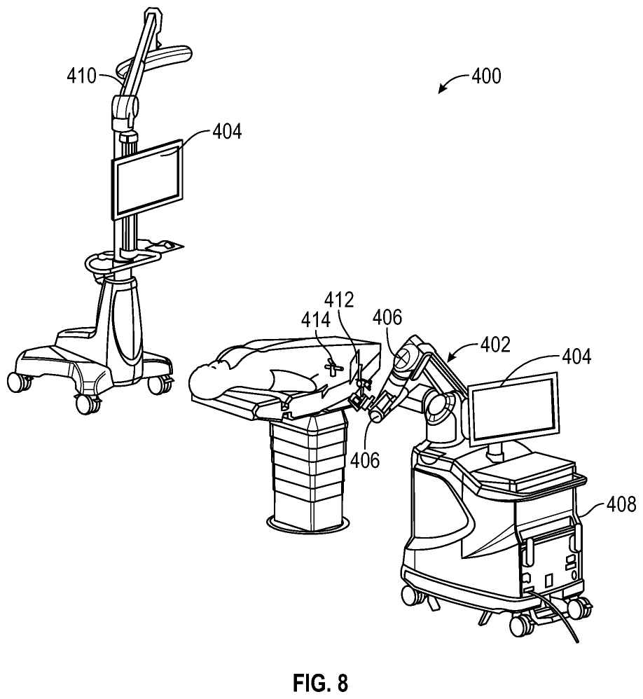

FIG. 8 illustrates a robotic surgical system 400, in accordance with at least one example of the present application. FIG. 6 is discussed with reference to the adaptor 100, the drill 102, and the impactor 104 shown in and described with regard to FIG. 1 above. The robotic surgical system 400 can include the robotic arm 402. The robotic arm 402 can be similar to the robotic arm 106 shown in and discussed with regard to FIG. 1 above. The robotic arm 402 can be controlled by a surgeon with various control devices or systems. For example, a surgeon can use a control system (e.g., a controller that is processor-implemented based on machine-readable instructions, which when implemented cause the robotic arm to move automatically or to provide force assistance to surgeon-guided movement) to guide the robotic arm 402. A surgeon can use anatomical imaging, such as displayed on display screens 404, to guide and position the robotic arm 402.

Anatomical imaging can be provided to the display screens 404 with various imaging sources, such as one or more cameras positioned on the robotic arm 402, or intraoperative fluoroscopy, such as a C-arm. The robotic arm 402 can include two or more articulating joints 406 capable of pivoting, rotating, or both, to provide a surgeon with wide range of adjustment options. The anatomical imaging, for example, can be imaging of internal patient anatomy within an incision. Such an incision can be made in a variety of positions on a patient. For example, in a hip arthroplasty procedure, the incision can be made in a hip region of a patient, such as to allow the impactor 104 (FIG. 1), when coupled to the robotic arm 402 to access a bone surface, or other anatomy of the patient.

The robotic arm 402 can include a computing system 408, which can also communicate with the display screens 404 and a tracking system 410. The tracking system 410 can be operated by the computing system 408 as a stand-alone unit. The computing system 408 can utilize the Polaris optical tracking system from Northern Digital, Inc. of Waterloo, Ontario, Canada. Additionally, the tracking system 410 can comprise the tracking system shown and described in Pub. No. US 2017/0312035, titled “Surgical System Having Assisted Navigation” to Brian M. May, which is hereby incorporated by this reference in its entirety. The tracking system 410 can monitor a plurality of tracking elements, such as tracking elements 412 and 414. The tracking elements 412 and 414 can be affixed to objects of interest, to track locations of multiple objects within a surgical field.

The tracking system 410 can function to create a virtual three-dimensional coordinate system within the surgical field for tracking patient anatomy, surgical instruments, or portions of the robotic arm 402 such as including the adaptor 100, the drill 102, or the impactor 104 when coupled thereto. One or more of the tracking elements 412 and 414 can be tracking frames including multiple IR reflective tracking spheres, or similar optically tracked marker devices. In an example, one or more of the tracking elements 412 and 414 can be placed on or adjacent one or more bones of patient. In other examples, one or more of the tracking elements 412 and 414 can be placed on the impactor 104 or on an implant to accurately track positions within the virtual coordinate system. In each instance, the tracking elements 412 and 414 can provide position data, such as a patient position, a bone position, a joint position, an implant position, a position of the robotic arm 402, or the like.

In the operation of some examples, the adaptor 100 can operatively couple the impactor 104 to the robotic arm 402, such as in preparation for a surgical arthroplasty procedure. The surgical procedure can be a hip arthroplasty; but can also be other types of joint replacement procedures. A surgeon can make an incision in a hip region of a patient. The robotic arm 402 can guide and position the impactor 104 to or within the incision. The impactor 104 can be guided to a bone surface of a patient using the robotic arm 402 in a cooperatively controlled mode utilizing robotic guidance, such as to position the head 105 (FIG. 1) of the impactor 104 at a surface of an implant positioned proximal to a bone surface. The drill 102 can then be selectively controlled to rotate the shaft 124 (FIG. 1) of the adaptor 100, such as to cause the adaptor 100 to impart an axial impaction force to the head 105.

The impactor 104 can thereby improve impaction of an implant into anatomy of a patient. In contrast to traditional methods using a manual impactor, the positioning and operation of the impactor 104, such as including striking the impactor with a mallet or setting an angle or trajectory of the impactor 104, can be easily and precisely carried out intra-procedurally with the adaptor 100 and the robotic arm 402. Further, the ability of the robotic arm 402 to be adjustably pivoted, rotated, or otherwise articulated intra-procedurally, either autonomously or cooperatively with the operator can help to increase the precision of implant positioning and impaction during an arthroplasty procedure.

For example, the robotic arm 402 can help to control the position and movement more precisely and steadily than a human hand and the adaptor 100 can impart a repetitive axial impaction force to the impactor 104 that is more consistent and predictable than a human hand. These benefits can enable a surgeon to complete a hip joint replacement procedure with improved accuracy and less fatigue; and provide a patient with shorter hospital stay and a reduced recovery time.

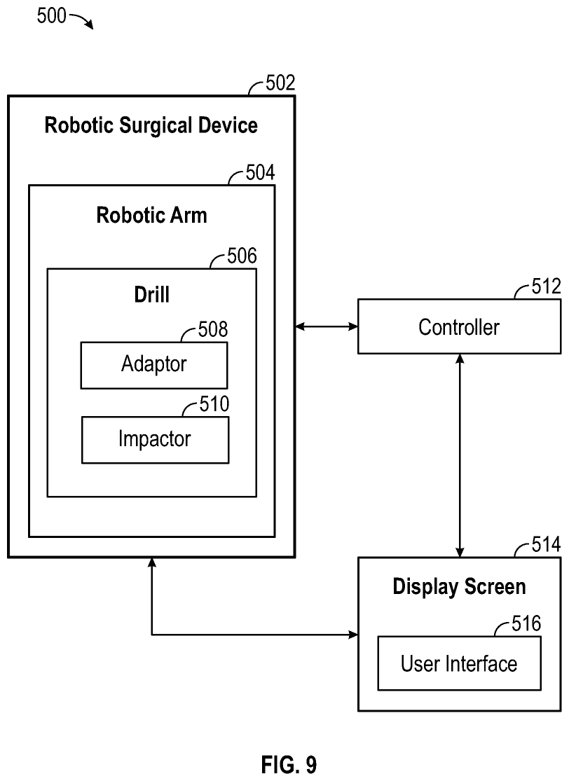

FIG. 9 illustrates a schematic view of a robotic surgical system 500 for robotically assisted impaction, in accordance with at least one example of the present application. The robotic surgical system 500 includes a robotic surgical device 502, which can include a robotic arm 504, and a drill 506. The drill 506 can be coupled to an adaptor 508 and an impactor 510. The robotic arm 504 can be similar to the robotic arm 106 discussed above with respect to FIG. 1, in that robotic arm 504 can be a movable and articulatable robotic arm. The drill 506, the adaptor 508, and the impactor 510 can be similar to the drill 102, the adaptor 100, and the impactor 104 shown in and discussed with respect to FIG. 1 above.

The robotic arm 504 can move autonomously in an example. In another example, the robotic arm 504 can provide a force assist to surgeon or user guided movements. In yet another example, a combination of autonomous movement and force assist movement can be performed by the robotic arm 504 (e.g., force assist for an initial movement, and autonomously moving a later movement). In an example, the robotic arm 504 can resist an applied force. For example, the robotic arm 504 can be programmed to stay within a particular range of locations or a particular position, move at a particular speed (e.g., resist a higher speed by resisting force), or the like.

The robotic surgical device 502 can output or receive data from a controller 512. The controller 512 can be implemented in processing circuitry (e.g., hardwired or a processor), a programmable controller, such as a single or multi-board computer, a direct digital controller (DDC), a programmable logic controller (PLC), a system on a chip, a mobile device (e.g., cell phone or tablet), a computer, or the like. In one example, the controller 512 can output information to a display screen 514. The display screen 514 can retrieve and display information from an imaging camera. The imaging camera can be physically positioned on the robotic surgical device 502, such as on the robotic arm 504, or on the drill 506, or alternatively, on the adaptor 508 or on impactor 510 coupled to the drill 506.

In an example, the display screen 514 can be used to display a user interface 516. In an example, the display screen 514 can be a touch screen display. In another example, the user interface 516 on the display screen 514 can provide lights, buttons, or switches. A user can thereby interact with the display screen 514 and the user interface 516 to input control commands, which can be relayed to the robotic surgical device 502 through the controller 512 to control the robotic surgical device 502. The robotic surgical system 500 can be used to perform all, or a portion of, a surgical procedure on a patient.

In the operation of some examples, a user can interact with the user interface 516 on the display screen 514 to power on the robotic surgical device 502. Power can be indicated by a light, for example, on the user interface 516, or on the robotic arm 504. When the robotic surgical device 502 is powered on, the user can operate the robotic arm 504 or the drill 506 by interacting with the display screen 514 and the user interface 516. In other examples, the drill 506 can be operated separately from the robotic surgical device 502 or the robotic arm 504, such as by operating a trigger of the drill 506.

The robotic surgical system 500 can be used to cut, impact, or otherwise shape a target bone surface of a patient, such as to prepare the bone surface to receive an implant by operating the drill 506 to cause the adaptor 508 to transmit an axial impaction force to the impactor 510. In an example, a cutting angle or trajectory of the impactor 510 can be changed intra-operatively, for example using the controller 512. The robotic arm 504 can thereby allow a user to respond to specific bone conditions of a patient, such as to improve an amount of a patient's bone that can be preserved during an arthroplasty procedure by increasing the consistency and precision of impaction of the bone surface. The bone penetration depth of the impactor can further be precisely controlled or otherwise limited using the robotic arm 504, in contrast to traditional manual or otherwise hand-held reamers.

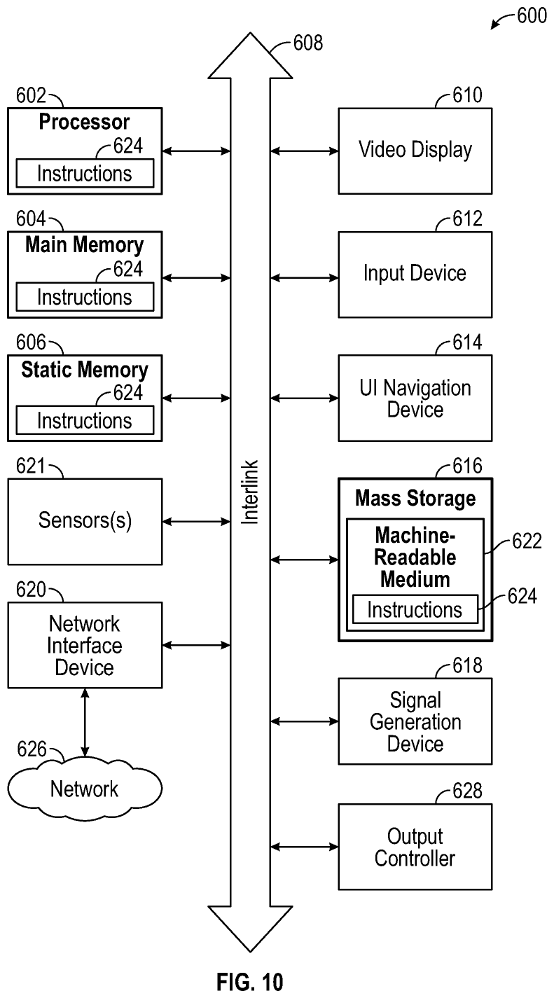

FIG. 10 illustrates a block diagram of an example machine upon which any one or more of the techniques discussed herein can be performed. In alternative embodiments, the machine 600 can operate as a standalone device or can be connected (e.g., networked) to other machines. In a networked deployment, the machine 600 can operate in the capacity of a server machine, a client machine, or both in server-client network environments. In an example, the machine 600 can act as a peer machine in peer-to-peer (P2P) (or other distributed) network environment.

The machine 600 can be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Machine (e.g., computer system) 600 can include a hardware processor 602 (e.g., a central processing unit (CPU), a graphics processing unit (GPU), a hardware processor core, or any combination thereof), a main memory 604 and a static memory 606, some or all of which can communicate with each other via an interlink 608 (e.g., bus)8. The machine 600 can further include a display unit 610, an alphanumeric input device 612 (e.g., a keyboard), and a user interface (UI) navigation device 614 (e.g., a mouse). In an example, the display unit 610, alphanumeric input device 612 and user interface (UI) navigation device 614 can be a touch screen display. The machine 600 can additionally include a storage device (e.g., drive unit) 616, a signal generation device 618 (e.g., a speaker), a network interface device 620, and one or more sensors 621, such as a global positioning system (GPS) sensor, compass, accelerometer, or other sensors. The machine 600 can include an output controller 628, such as a serial (e.g., Universal Serial Bus (USB), parallel, or other wired or wireless (e.g., infrared (IR), near field communication (NFC), etc.) connection to communicate or control one or more peripheral devices (e.g., a printer, card reader, etc.).

The storage device 616 can include a machine readable medium 622 on which is stored one or more sets of data structures or instructions 624 (e.g., software) embodying or utilized by any one or more of the techniques or functions described herein. The instructions 624 can also reside, completely or at least partially, within the main memory 604, within static memory 606, or within the hardware processor 602 during execution thereof by the machine 600. In an example, one or any combination of the hardware processor 602, the main memory 604, the static memory 606, or the storage device 616 can constitute machine readable media.

While the machine readable medium 622 is illustrated as a single medium, the term “machine readable medium” can include a single medium or multiple media (e.g., a centralized or distributed database, or associated caches and servers) configured to store the one or more instructions 624. The term “machine readable medium” can include any medium that is capable of storing, encoding, or carrying instructions for execution by the machine 600 and that cause the machine 600 to perform any one or more of the techniques of the present disclosure, or that is capable of storing, encoding or carrying data structures used by or associated with such instructions. Non-limiting machine-readable medium examples can include solid-state memories, and optical and magnetic media.

The instructions 624 can further be transmitted or received over a communications network 626 using a transmission medium via the network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others.

In an example, the network interface device 620 can include one or more physical jacks (e.g., Ethernet, coaxial, or phone jacks) or one or more antennas to connect to the communications network 626. In an example, the network interface device 620 can include a plurality of antennas to wirelessly communicate using at least one of single-input multiple-output (SIMO), multiple-input multiple-output (MIMO), multiple-input single-output (MISO) techniques. The term “transmission medium” shall be taken to include any intangible medium that is capable of storing, encoding or carrying instructions for execution by the machine 600, and includes digital or analog communications signals or other intangible medium to facilitate communication of such software.

The foregoing systems and devices, etc. are merely illustrative of the components, interconnections, communications, functions, etc. that can be employed in carrying out examples in accordance with this disclosure. Different types and combinations of sensor or other portable electronics devices, computers including clients and servers, implants, and other systems and devices can be employed in examples according to this disclosure.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided.

Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein. In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure.

This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

### Notes and Examples

The above description and the drawings sufficiently illustrate specific examples to enable those skilled in the art to practice them. Other examples may incorporate structural, process, or other changes. Portions and features of some examples may be included in, or substituted for, those of other examples. Examples set forth in the claims encompass all available equivalents of those claims. The following, non-limiting examples, detail certain aspects of the present subject matter to solve the challenges and provide the benefits discussed herein, among others.

Example 1 is an adaptor configured to receive a rotational force from a surgical drill to impart an axial impaction force to a surgical impactor connectable to a robotic arm, the adaptor comprising: a proximal portion defining a longitudinal axis and including a first end portion and a second end portion, the proximal portion defining a body bore extending between the first end portion and the second end portion along the longitudinal axis, the second end portion including a plurality of first projections extending proximally therefrom into the body bore; a distal portion connected to the proximal portion and insertable into the surgical impactor to locate the distal portion with respect to the surgical impactor; a shaft extending into the body bore, the shaft engageable with the surgical drill to receive the rotational force; a driving body translatable within the body bore along the longitudinal axis and connected to the shaft, the driving body including a plurality of second projections extending distally therefrom, the second projections engageable with the first projections to translate the driving body distally relative to the shaft in response to rotation of the shaft; and a biasing element located within the body bore engaged with the proximal portion and the driving body to bias the driving body distally.

In Example 2, the subject matter of Example 1 includes, wherein the proximal portion defines an outer surface having a diameter greater than a diameter of an outer surface of the distal portion.

In Example 3, the subject matter of Examples 1-2 includes, wherein the second end portion of the proximal portion is engageable with the surgical impactor to limit distal translation of the adaptor within the surgical impactor.

In Example 4, the subject matter of Examples 1-3 includes, wherein the first end portion defines a proximal bearing for the shaft.

In Example 5, the subject matter of Examples 1-4 includes, a pair of opposing protrusions extending radially outward from a body surface of the shaft.

In Example 6, the subject matter of Example 5 includes, wherein the driving body includes a proximal surface and a distal surface, the driving body defining a shaft bore extending longitudinally therebetween and configured to receive a portion of the shaft.

In Example 7, the subject matter of Example 6 includes, wherein the driving body defines a slot extending longitudinally through the proximal surface of the driving body and intersecting the shaft bore, the slot configured to translatably receive the pair of protrusions to transfer torque from the shaft to the driving body.

In Example 8, the subject matter of Examples 1-7 includes, wherein the first end portion of the proximal portion includes a taper extending distally into the body bore to support the biasing element.

In Example 9, the subject matter of Examples 1-8 includes, wherein each of the first projections includes an angled surface rotatably engageable angled surfaces of one the second plurality of projections to cause proximal translation of the driving body within the body bore, and wherein each angled surface of the second projections is complementary to each angled surface of each of the first projections.

Example 10 is an adaptor configured to receive a rotational force from a surgical drill to impart an axial impaction force to a surgical impactor connectable to a robotic arm, the adaptor comprising: a proximal portion defining a longitudinal axis and including a first end portion and a second end portion, the proximal portion defining a body bore extending longitudinally between the first end portion and the second end portion, the second end including a plurality of first projections extending proximally therefrom into the body bore, and a distal portion connected to the proximal portion and insertable in the surgical impactor to locate the distal portion with respect to the surgical impactor; a shaft extending into the body bore and engageable with the surgical drill to receive the rotational force; a driving body translatable within the body bore along the longitudinal axis and connected to the shaft, the driving body including a plurality of second projections extending distally therefrom, the second projections rotatably engageable with the first projections to translate the driving body distally relative to the shaft in response to rotation of the shaft to deliver the axial impaction force to the surgical impactor in response to rotation of the shaft, and wherein the driving body defines a shaft bore extending longitudinally axially between a proximal surface and a distal surface thereof, the shaft bore configured to translatably receive a portion of the shaft to allow proximal and distal translation of the driving body relative to the shaft; and a biasing element located within the body bore engaged with the proximal portion and the driving body to bias the driving body distally.

In Example 11, the subject matter of Example 10 includes, wherein a first portion of the shaft includes a facet engageable with the surgical drill to prevent relative rotation between the shaft and the surgical drill.

In Example 12, the subject matter of Example 11 includes, wherein a second portion of the shaft is hemispherically shaped.

In Example 13, the subject matter of Example 12 includes, wherein the first end portion of the proximal portion comprises a removable cap defining an aperture extending therethrough.

In Example 14, the subject matter of Example 13 includes, wherein the removable cap includes a proximal bearing located within the aperture of the removable cap, the bearing configured to reduce rotational friction between the shaft and the removable cap.

In Example 15, the subject matter of Example 14 includes, wherein the first end of the proximal portion defines a plurality of threaded bores and the removable cap defines a plurality of apertures, wherein the plurality of threaded bores and the plurality of apertures are configured to concurrently receive a plurality of fasteners to secure the removable cap to the proximal portion.

In Example 16, the subject matter of Examples 10-15 includes, wherein the shaft includes a protrusion extending radially outward beyond an outer surface of the shaft, and wherein the driving body defines a slot extending longitudinally through the proximal surface of the driving body and intersecting the shaft bore, the slot configured to translatably receive the protrusion to allow proximal and distal translation of the driving body relative to the shaft.

In Example 17, the subject matter of Examples 10-16 includes, wherein the first projections and the second projections each include three projections, wherein a radial surface of each of the first projections and the second projections is spaced apart from a radial surface of each adjacent projection of the first projections and the second projections by about 80 degrees.

In Example 18, the subject matter of Examples 10-17 includes, wherein the first projections and the second projections each include four projections, wherein a radial surface of each of the first projections and the second projections is spaced apart from a radial surface of each adjacent projection of the first projections and the second projections by about 50 degrees.

Example 19 is an impaction adaptor connectable to a surgical drill and a surgical impactor, the impaction adaptor comprising: a body comprising: a proximal portion defining a body bore and including a first plurality of projections; and a distal portion connected to the proximal portion and insertable into the surgical impactor; a shaft located at least partially within the body bore and engageable with the surgical drill to be driven to rotate within the body bore; a biasing element located within the body bore and engaged with the proximal portion of the body; and a driving body located at least partially within the body bore, the driving body secured to the shaft and engaged with the biasing element, the driving body including a plurality of second projections rotatably engageable with the first projections to cause translation of the driving body relative to the body to deliver an impaction force to the surgical impactor in response to rotation of the shaft.

In Example 20, the subject matter of Example 19 includes, wherein the body defines a longitudinal axis, and the body bore extends longitudinally axially between a first end portion and a second end portion of the proximal portion.

In Example 21, the subject matter of Example 20 includes, wherein the second end portion of the proximal portion is engageable with the surgical impactor to limit distal translation of the impaction adaptor with respect to the surgical impactor.

In Example 22, the subject matter of Examples 20-21 includes, wherein the first end portion of the proximal portion defines an aperture extending through the first end portion of the proximal portion, the shaft extending through the aperture into the body bore.

In Example 23, the subject matter of Example 22 includes, wherein the first end portion of the proximal portion comprises a removable cap defining a plurality of apertures and the proximal portion defines a plurality of threaded bores, and wherein the plurality of threaded bores and the plurality of apertures are configured to concurrently receive a plurality of fasteners to secure the removable cap to the proximal portion.

In Example 24, the subject matter of Examples 20-23 includes, wherein a first end portion defines a proximal bearing for the shaft.

In Example 25, the subject matter of Examples 19-24 includes, wherein the proximal portion defines an outer surface having a diameter greater than a diameter of an outer surface of the distal portion.

In Example 26, the subject matter of Examples 19-25 includes, wherein the driving body includes a proximal surface, a distal surface, and defines a shaft bore extending longitudinally axially therebetween, the shaft bore configured to translatably receive a portion the shaft, and a slot extending longitudinally through the proximal surface of the driving body and intersecting the shaft bore, the slot configured to translatably receive a pair of protrusion extending radially outward from the shaft to allow proximal and distal translation of the driving body relative to the shaft.

In Example 27, the subject matter of Examples 19-26 includes, wherein each of the first projections includes an angled surface rotatably engageable with angled surfaces of one the second plurality of projections to cause proximal translation of the driving body within the body bore, and wherein each angled surface of the second projections is complementary to each angled surface of each of the first projections.

In Example 28, the subject matter of Example 27 includes, wherein the first projections and the second projections each include three projections, wherein a radial surface of each of the first projections and the second projections is spaced apart from a radial surface of each adjacent projection of the first projections and the second projections by about 97 degrees.

In Example 29, the subject matter of Examples 27-28 includes, wherein the first projections and the second projections each include four projections, wherein a radial surface of each of the first projections and the second projections is spaced apart from a radial surface of each adjacent projection of the first projections and the second projections by about 67 degrees.

Example 30 is at least one machine-readable medium including instructions that, when executed by processing circuitry, cause the processing circuitry to perform operations to implement of any of Examples 1-29.

Example 31 is an apparatus comprising means to implement of any of Examples 1-29.

Example 32 is a system to implement of any of Examples 1-29.

Example 33 is a method to implement of any of Examples 1-29.

Example 34 is a method of imparting an axial impaction force to a surgical impactor, the method comprising: inserting a distal portion of an adaptor into the surgical impactor coupled to the surgical robotic arm; coupling a first portion of a shaft of the adaptor to the surgical drill; and activating the surgical drill to cause the adaptor to impact an axial impaction force to the surgical impactor.

In Example 35, the subject matter of Example 34 includes, wherein the method first comprises coupling the surgical impactor to a surgical robotic arm.

In Example 36, the subject matter of Examples 34-35 includes, wherein activating the surgical drill includes controlling movement of the surgical robotic arm to position the surgical impactor and the surgical drill.

Example 37 is a method of converting a surgical system configured to ream bone with a rotatable cutting head to a surgical system configured to impact bone with a translatable cutting head or implant, the method comprising: replacing the rotatable cutting head of a surgical device connected to a robotic arm with the axially translatable cutting head or implant; decoupling a surgical drill from the surgical device; inserting a distal portion of an adaptor into a channel of the surgical device, the adaptor configured to transform a rotational force generated by the surgical drill into an axial impaction force transmittable to the surgical device; and coupling a first portion of a shaft of the adaptor to the surgical drill.

In Example 38, the method of Example 37 further comprises wherein replacing the rotatable cutting head of a surgical device connected to a robotic arm with the axially translatable cutting head or implant includes disconnecting the rotatable cutting head from a rod translatably and rotatably received within the channel and connecting the translatable cutting head or implant to the rod; wherein decoupling the surgical drill from the surgical device includes decoupling a chuck of the surgical drill from the rod; and wherein inserting the distal portion of the adaptor into the channel of the surgical device includes positioning the distal portion of the adaptor in contact with the rod.

In Example 39, the method of Example 38 includes, wherein the implant is a replacement acetabular cup.

Example 40 is at least one machine-readable medium including instructions that, when executed by processing circuitry, cause the processing circuitry to perform operations to implement of any of Examples 1-39.

Example 41 is an apparatus comprising means to implement of any of Examples 1-39.

Example 42 is a system to implement of any of Examples 1-39.

What is claimed is:

1. An adaptor configured to receive a rotational force from a surgical drill to impart an axial impaction force to a surgical impactor connectable to a robotic arm, the adaptor comprising:
a proximal portion defining a longitudinal axis and including a first end portion and a second end portion, the proximal portion defining a body bore extending between the first end portion and the second end portion along the longitudinal axis, the second end portion including a plurality of first projections extending proximally therefrom into the body bore;
a distal portion connected to the proximal portion and insertable into the surgical impactor to locate the distal portion with respect to the surgical impactor;
a shaft extending into the body bore, the shaft engageable with the surgical drill to receive the rotational force;
a driving body translatable within the body bore along the longitudinal axis and connected to the shaft, the driving body including a plurality of second projections extending distally therefrom, the second projections engageable with the first projections to translate the driving body distally relative to the shaft in response to rotation of the shaft; and
a biasing element located within the body bore engaged with the proximal portion and the driving body to bias the driving body distally.

2. The adaptor of claim 1, wherein the proximal portion defines an outer surface having a diameter greater than a diameter of an outer surface of the distal portion.

3. The adaptor of claim 1, wherein the second end portion of the proximal portion is engageable with the surgical impactor to limit distal translation of the adaptor within the surgical impactor.

4. The adaptor of claim 1, wherein the first end portion defines a proximal bearing for the shaft.

5. The adaptor of claim 1, further comprising:
a pair of opposing protrusions extending radially outward from a body surface of the shaft.

6. The adaptor of claim 5, wherein the driving body includes a proximal surface and a distal surface, the driving body defining a shaft bore extending longitudinally therebetween and configured to receive a portion of the shaft.

7. The adaptor of claim 6, wherein the driving body defines a slot extending longitudinally through the proximal surface of the driving body and intersecting the shaft bore, the slot configured to translatably receive the pair of protrusions to transfer torque from the shaft to the driving body.

8. The adaptor of claim 1, wherein the first end of the proximal portion includes a taper extending distally into the body bore to support the biasing element.

9. The adaptor of claim 1, wherein each of the first projections includes an angled surface rotatably engageable angled surfaces of one the second plurality of projections to cause proximal translation of the driving body within the body bore, and wherein each angled surface of the second projections is complementary to each angled surface of each of the first projections.

10. An adaptor configured to receive a rotational force from a surgical drill to impart an axial impaction force to a surgical impactor connectable to a robotic arm, the adaptor comprising:
a proximal portion defining a longitudinal axis and including a first end portion and a second end portion, the proximal portion defining a body bore extending longitudinally between the first end portion and the second end portion, the second end including a plurality of first projections extending proximally therefrom into the body bore, and
a distal portion connected to the proximal portion and insertable in the surgical impactor to locate the distal portion with respect to the surgical impactor;
a shaft extending into the body bore and engageable with the surgical drill to receive the rotational force;
a driving body translatable within the body bore along the longitudinal axis and connected to the shaft, the driving body including a plurality of second projections extending distally therefrom, the second projections rotatably engageable with the first projections to translate the driving body distally relative to the shaft in response to rotation of the shaft to deliver the axial impaction force to the surgical impactor in response to rotation of the shaft, and wherein the driving body defines a shaft bore extending longitudinally axially between a proximal surface and a distal surface thereof, the shaft bore configured to translatably receive a portion of the shaft to allow proximal and distal translation of the driving body relative to the shaft; and
a biasing element located within the body bore engaged with the proximal portion and the driving body to bias the driving body distally.

11. The adaptor of claim 10, wherein a first portion of the shaft includes a facet engageable with the surgical drill to prevent relative rotation between the shaft and the surgical drill.

12. The adaptor of claim 11, wherein a second portion of the shaft is hemispherically shaped.

13. The adaptor of claim 12, wherein the first end of the proximal portion comprises a removable cap defining an aperture extending therethrough.

14. The adaptor of claim 13, wherein the removable cap includes a proximal bearing located within the aperture of the removable cap, the bearing configured to reduce rotational friction between the shaft and the removable cap.

15. The adaptor of claim 14, wherein the first end of the proximal portion defines a plurality of threaded bores and the removable cap defines a plurality of apertures, wherein the plurality of threaded bores and the plurality of apertures are configured to concurrently receive a plurality of fasteners to secure the removable cap to the proximal portion.

16. The adaptor of claim 10, wherein the shaft includes a protrusion extending radially outward beyond an outer surface of the shaft, and wherein the driving body defines a slot extending longitudinally through the proximal surface of the driving body and intersecting the shaft bore, the slot configured to translatably receive the protrusion to allow proximal and distal translation of the driving body relative to the shaft.

17. The adaptor of claim 10, wherein the first projections and the second projections each include three projections, wherein a contacting surface of each of the first projections and the second projections is spaced apart from a radial surface of each adjacent projection of the first projections and the second projections by about 97 degrees.

18. The adaptor of claim 10, wherein the first projections and the second projections each include four projections, wherein a contacting surface of each of the first projections and the second projections is spaced apart from a radial surface of each adjacent projection of the first projections and the second projections by about 67 degrees.

19. An impaction adaptor connectable to a surgical drill and a surgical impactor, the impaction adaptor comprising:
a body comprising:
a proximal portion defining a body bore and including a first plurality of projections; and
a distal portion connected to the proximal portion and insertable into the surgical impactor;
a shaft located at least partially within the body bore and engageable with the surgical drill to be driven to rotate within the body bore;
a biasing element located within the body bore and engaged with the proximal portion of the body; and
a driving body located at least partially within the body bore, the driving body secured to the shaft and engaged with the biasing element, the driving body including a plurality of second projections rotatably engageable with the first projections to cause translation of the driving body relative to the body to deliver an impaction force to the surgical impactor in response to rotation of the shaft.

20. The impaction adaptor of claim 19, wherein the body defines a longitudinal axis, and the body bore extends longitudinally axially between a first end portion and a second end portion of the proximal portion.

21. The impaction adaptor of claim 20, wherein the second end portion of the proximal portion is engageable with the surgical impactor to limit distal translation of the impaction adaptor with respect to the surgical impactor.

22. The impaction adaptor of claim 20, wherein the first end portion of the proximal portion defines an aperture extending through the first end portion of the proximal portion, the shaft extending through the aperture into the body bore.

23. The impaction adaptor of claim 22, wherein the first end portion of the proximal portion comprises a removable cap defining a plurality of apertures and the proximal portion defines a plurality of threaded bores, and wherein the plurality of threaded bores and the plurality of apertures are configured to concurrently receive a plurality of fasteners to secure the removable cap to the proximal portion.
